(19) United States
(12) Patent Application Publication
(10) Pub. No.: US 2009/0305379 A1
(43) Pub. Date: Dec. 10, 2009

(54) DIGESTER SYSTEM

(76) Inventors: Matthew W. Johnson, Lake Crystal, MN (US); Dennis J. Johnson, Windom, MN (US)

Correspondence Address: FAEGRE & BENSON LLP;PATENT DOCKETING - INTELLECTUAL PROPERTY, 2200 WELLS FARGO CENTER, 90 SOUTH SEVENTH STREET, MINNEAPOLIS, MN, 55402-3901, US

(21) Appl. No.: 12/134,435

(22) Filed: Jun. 6, 2008

(51) Int. Cl. C12P 1/04; C12M 1/00; C12M 1/36

(52) U.S. Cl. 435/170; 435/290.4; 435/286.2; 435/286.1; 435/286.5; 435/290.1

(57) ABSTRACT

A manure mixture within an anaerobic digestion tank stratifies to form a liquid effluent layer and a sludge layer. Liquid effluent from the liquid effluent layer is withdrawn from the tank through a height adjustable valve. The height adjustable valve is adapted to automatically adjust the position of its intake end within the liquid effluent layer in response to the level of the sludge layer detected by a sludge meter located within the tank. Liquid effluent withdrawn from the tank is passed through a heat exchange system including at least one heat exchanger. Heat from the heat exchanger is transferred to the liquid effluent to produce heated liquid effluent. The heated liquid effluent is reintroduced back into the digestion tank such that the temperature of the manure mixture within the tank is maintained within a suitable temperature range for anaerobic digestion of the manure mixture. Additionally, the heated liquid effluent is sprayed in an upwards direction so as to effect mixing of the manure mixture within the tank.

### TECHNICAL FIELD

The present invention relates to a system and a method for converting agricultural waste into biogas. More specifically, the present invention relates to a system and method utilizing anaerobic digestion for converting animal waste into methane gas.

### BACKGROUND

Animal waste poses a significant problem in the poultry, swine, and dairy industries. In addition to foul odors, animal waste from animal raising or processing operations can contribute to decreases in the air and water quality in the surrounding farms and communities. Anaerobic digestion has been used to convert animal and other agriculture waste into biogas and other useful byproducts, decreasing the impact on the surrounding environment and making the waste a useful, renewable resource.

The anaerobic digestion process has been utilized to treat and remove organic compounds from waste products such as sewage, sewage sludge, chemical wastes, food processing wastes, agricultural residues, animal wastes, including manure and other organic waste and material. Organic waste materials are fed into an anaerobic digestion reactor or tank which is sealed to prevent entrance of oxygen. Under these air free or “anoxic” conditions, anaerobic bacteria digests the waste. Anaerobic digestion may be carried out in a single reactor or in multiple reactors of the two-stage or two-phase configuration. Heat is normally added to the reactor or reactors to maintain adequate temperatures for thermophilic or mesophilic bacteria which accomplish the breakdown of the organic material.

The products or effluent from anaerobic digestion typically include: a gas phase containing carbon dioxide, methane, ammonia, and trace amounts of other gases, such as hydrogen sulfide, which in total comprise what is commonly called biogas; a liquid phase containing water, dissolved ammonia nitrogen, nutrients, organic and inorganic chemicals; and a colloidal or suspended solids phase containing undigested organic and inorganic compounds, and synthesized biomass or bacterial cells within the effluent liquid. The biogas can be collected and used for a wide variety of applications including as an energy source for the digestion process itself. Maintaining conditions for optimal digestion of the waste facilitates an efficient digestion process.

### SUMMARY

According to various embodiments, the present invention is a digestion system for converting agriculture waste to biogas including a digestion tank, a heat exchange system, a water recirculation system, a biogas collection system, and a biogas conditioning system. The digestion tank, heat exchange system, water recirculation system, biogas collection system, and biogas conditioning system are coupled to and controlled by a main controller. The main controller controls the interactions between the various systems of the digestion system.

According to various embodiments, the digestion tank includes a first end and a second end and a predetermined level of a manure mixture to be digested. The manure mixture includes a liquid effluent layer having a liquid level and a sludge layer having a solids level and a quantity of anaerobic bacteria adapted to digest the manure mixture to produce biogas. A headspace is defined above the predetermined level of the manure mixture within the tank. Biogas is collected in the headspace. According to some embodiments, the digestion tank also includes a height adjustable valve including an intake end configured to withdraw liquid effluent from the liquid effluent layer out of the digestion tank.

According to various embodiments, the heat exchange system includes at least one heat exchanger and is fluidly coupled to the digestion tank via a first recirculation line and a second recirculation line. Liquid effluent from the liquid effluent layer flows through the heat exchange system via the first recirculation line and is returned to the digestion tank via the second recirculation line.

According to various embodiments, the water circulation system includes at least one water heater and at least one pump. The water circulation system pumps hot water to provide heat to the heat exchanger or multiple heat exchangers of the heat exchange system.

According to various embodiments, the biogas collection system includes a pressure relief valve, a flow meter, and a positive displacement blower. The biogas collection system regulates a level of biogas collected in the headspace of the digestion tank, and transfers biogas from the digestion tank to the biogas conditioning system as needed or desired. According to some embodiments, the biogas conditioning system is configured to remove moisture and impurities from the biogas.

According to other embodiments, the present invention is a digestion tank assembly including a digestion tank and a height adjustable valve. According to various embodiments, the digestion tank includes: a first end and a second end, a predetermined level of a manure mixture to be digested, the manure mixture including a liquid effluent layer having a liquid level and a sludge layer having a solids level; a quantity of anaerobic bacteria adapted to digest the manure mixture to produce biogas, and a headspace defined above the predetermined level of the manure mixture within the tank. Biogas is collected in the headspace defined within the digestion tank. According to various embodiments, the height adjustable valve is coupled to and located within the tank and includes an intake end configured to withdraw liquid effluent out of the digestion tank. The intake end is adapted to move in a vertical direction between at least a first position and a second position such that the intake end is maintained within the liquid effluent layer and above the sludge layer within the digestion tank.

According to yet other embodiments, the present invention is a process for converting agricultural waste to biogas. In various embodiments, the process includes: transferring fresh waste from a waste reception area to an anaerobic digestion tank including a sufficient quantity of anaerobic bacteria to digest the waste to produce a manure mixture having a predetermined level and including a liquid effluent layer having a liquid level and a sludge layer having a solids level and biogas; determining the solids level of the sludge layer within the digestion tank; maintaining an intake end of a valve configured to withdraw liquid effluent from the digestion tank within the liquid effluent layer and above the sludge layer; withdrawing liquid effluent from the liquid effluent layer; transferring the liquid effluent through a heat exchange system fluidly coupled to the digestion tank to produce heated liquid effluent; and maintaining a temperature of the manure mixture within the digestion tank by returning the heated liquid effluent to the digestion tank and spraying the heated liquid effluent in an upwards direction to mix the manure mixture within the tank.

### BRIEF DESCRIPTION OF THE DRAWINGS

FIG. 1 is a diagrammatic flow chart showing the major steps and components of a digester system according to one embodiment of the present invention.

FIG. 2A is a side, schematic view of a digestion tank according to one embodiment of the present invention.

FIG. 2B is a top, schematic view of the digestion tank shown in FIG. 2A.

FIG. 3 is a schematic block diagram of a portion of a digestion system according to one embodiment of the present invention.

FIG. 4 is a side schematic view of the digestion tank, as shown in FIGS. 2A and 2B, according to one embodiment of the present invention.

FIG. 5 is a schematic block diagram of a portion of the digestion system according to one embodiment of the present invention.

FIG. 6 is a schematic view of moisture knockout provided in accordance with an embodiment of the present invention.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that other embodiments may be utilized and that structural changes may be made without departing from the scope of the present invention. Therefore, the following detailed description is not to be taken in a limiting sense, and the scope of the present invention is defined by the appended claims and their equivalents.

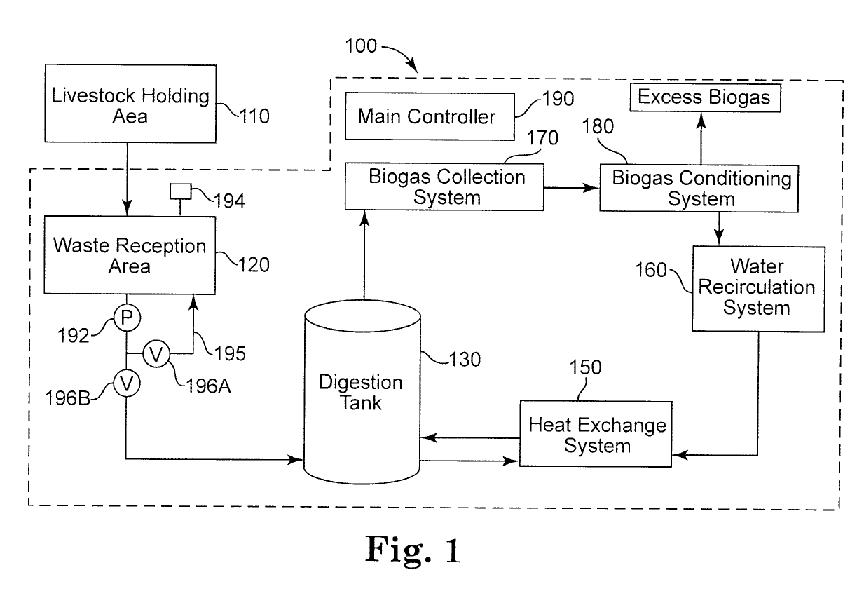

FIG. 1 is a diagrammatic flow chart of a digestion system 100, according to various embodiments of the present invention. The digestion system 100 can be located near and fluidly coupled to a livestock holding area 10. As shown in FIG. 1, the digestion system 100 includes: a waste reception area 120, a digestion tank 130; a heat exchange system 150, a water circulation system 160; a biogas collection system 170; and a biogas conditioning system 180. The various functions of the components of the digestion system 100 are controlled through a main controller 190. While the digestion system 100 is generally described as it relates to agriculture facilities it is generally recognized by those of skill in the art that the digestion system 100 is applicable to other waste processing facilities. Additionally, the digestion system 100 can be modular which facilitates its use in a variety of small and larger scale applications.

As shown in FIG. 1, animal manure is collected and transferred from the livestock holding area 110 to the waste reception area 120 where it is pooled prior to beginning the digestion process. Depending upon the type of waste to be digested, the solids content of the animal manure can vary from about 1% to about 50% (v/v %). According to one embodiment, the total solids content of the animal manure to be digested should range from about 3% to about 18% (v/v %). If the percent of total solids in the waste is greater than approximately 18% (v/v %), the manure may be mixed with water or another aqueous mixture until the manure is of the desired consistency suitable for digestion. In some cases, as with poultry facilities or other waste producing facilities, the waste stream may be thick and may require the addition of an aqueous mixture such that the solids content is reduced and the waste stream is in a pumpable form. Additionally, agitation may be required to break down the solids prior to delivery to the digestion tank. Agitation of the waste within the reception area can be facilitated by a mixer or propeller (not shown) located within the waste reception area 120. In other embodiments, the total solids content of the waste may be reduced by recirculating the waste stream from the digestion tank 130 back into the waste reception area 120. This is accomplished via one or more motorized ball valves 192 which are placed in the line of flow such that in operation they are configured to redirect the flow of waste from the digestion tank 130 to the waste reception area 120.

According to various embodiments, the waste reception area 120 includes a pump 192, at least one ultrasonic level detector 194 for detecting a level of manure in the waste reception area 120, a recirculation line 195, and one or more valves 196A and 196B adapted to release the manure from the waste reception area 120 to the digestion tank 130.

The pump 192 can be any suitable pump known to those of skill the art. In some embodiments, the pump 192 transfers the waste from the reception area 120 directly to the digestion tank 130. In other embodiments, the pump 192 re-circulates the waste through the recirculation line 195 prior to transferring the waste to the digestion tank 130.

The ultrasonic level detector 194 detects the level of waste in the waste reception area 194 and sends this information to the main controller 190. An exemplary ultrasonic level detector is the Drexelbrook Ultrasonic Level Detector US11. It is generally recognized by those of ordinary skill in the art that other detectors capable of detecting the level of the liquid effluent layer may also be employed.

According to some embodiments, the valve(s) 196A and 196B are motorized ball valves. In some embodiments, the valves 196 may be operated to open and close at specific time intervals using a timing mechanism controlled by the main controller 190 to allow waste to flow through either the recirculation line 195 or directly from the reception area 120 to the digestion tank 130. For example, when valve 196B is closed and 196A is open, the waste is recirculated back to the reception area 120 via the recirculation line 195. Conversely, when valve 196B is open and 196A is closed the waste flows directly from the reception area 120 to the digestion tank 130. In some embodiments, the valve(s) 196A and B are controlled to open and close through the main controller 190 in response to waste level determinations made by the ultrasonic level detector 194. For example, when the manure inside the waste reception area 120 reaches a level indicative of a potential overflow, as determined by the level detector 194, the main controller 190 signals valve 196B to open and 196A to close to allow the manure to flow from the waste reception area 120 to the digestion tank 130. This feature assists in protecting against overflow of the reception area 120. Similarly, if the manure level detected by the level detector 194 is too low, the main controller 190 can send an output command to the pump 192 to cease pumping, overriding any pre-programmed timing intervals. When the manure level reaches an acceptable minimal level as determined by the level detector, the pump 192 can resume and the valves 196A and 196B can open and close on their regularly programmed intervals.

In some embodiments, where the solids content of the waste stream is low such as with hog facilities, the reception area 120 may exclude the recirculation loop 195 and may be directly connected to the digestion tank 130. The waste may be pumped directly from the reception area 120 to the digestion tank 130. The waste may be delivered to the digestion tank either continuously or in a batch-wise process. In some embodiments, the pump may be operated on a timing mechanism.

Waste from the waste reception area 120 is transferred to the digestion tank 130 where it undergoes anaerobic digestion. The influent waste enters the digestion tank 130 through an inlet located near the bottom of the tank 130. Depending upon the application or the size of the operation, fresh influent can be added to the digestion tank 130 in a batch or continuous flow process. Digested effluent is released from the digestion tank 130 in equal proportion to fresh influent transferred from the reception area 120 to the tank.

Liquid effluent waste from the digestion tank 130 is drawn out of the tank and into the heat exchange system 150, where it is heated. The water circulation system 160 provides heat to the heat exchange system 150. The heated effluent is then recirculated from the heat exchange system 150 back into the digestion tank 130. The heated effluent helps to maintain the temperature of the mixture inside the tank 130 at a temperature range sufficient for an efficient anaerobic digestion process to occur, while at the same time allowing the waste mixture inside the tank 130 to be efficiently mixed. By providing a system that facilitates efficient heating and mixing of the manure mixture within the digestion tank, the residency time of the manure mixture within the tank may be decreased. Reducing the residency time of the manure mixture in the tank, allows for a larger volume of waste to be processed without increasing the overall size of the tank and ancillary components. According to one embodiment, the digestion system 100 is configured to re-circulate more than one tank volume per day. According to another embodiment, the digestion system 100 system is adapted to re-circulate up to about three to about six tank volumes per day. According to other embodiments, the tank 130 is modular such that two or more tanks can be operated in parallel to meet the waste processing demands of larger agricultural operations and industrial or municipal waste processing facilities.

Biogas produced from the anaerobic digestion process is drawn out of the digestion tank 130 by the biogas collection system 170 from which the biogas is then passed through the biogas conditioning system 180. In one embodiment, the conditioned biogas is then used to provide energy to the water recirculation system 160 which in turn provides heat to the heat exchange system 150. The excess biogas 198 can be collected and used to provide energy to other farm components, such as generator (not shown), used to provide energy to the water recirculation system 160, or burned off via a flare. Additionally, the excess biogas 198 can be used to offset the energy use of industrial operations. For example, the excess biogas 198 can be used to offset the energy use of an ethanol plant or other production facility.

According to various embodiments, the various components of the digestion system 100 are controlled by the main controller 190. The main controller 190 monitors a series of inputs received from each of the digestion system components and is programmed to respond with a series of outputs based on the information that is received. According to one embodiment, the main controller 190 includes a PID control loop which attempts to correct any difference between a measured process variable received from a digestion system component and a predetermined value by calculating and then outputting a corrective action that can adjust the process accordingly. According to various embodiments, the main controller 190 also includes a data management and storage device (not shown) such that all data received from the various system components is saved and can be analyzed to adjust the process parameters of the digester system. Additionally, the main controller 190 is adapted to be connected to the internet such that all input values and output values can be remotely monitored, and any necessary adjustments to the operation made without visiting the facility where the digestion system 100 is located. According to one embodiment of the present invention, the main controller 190 is a Honeywell hybrid loop and logic controller.

According to various embodiments, the heat exchange system 150, water circulation system 160, biogas collection system 170, biogas conditioning system 180, and main controller 190 can be located together within a building (not shown) provided separately from the digestion tank 130.

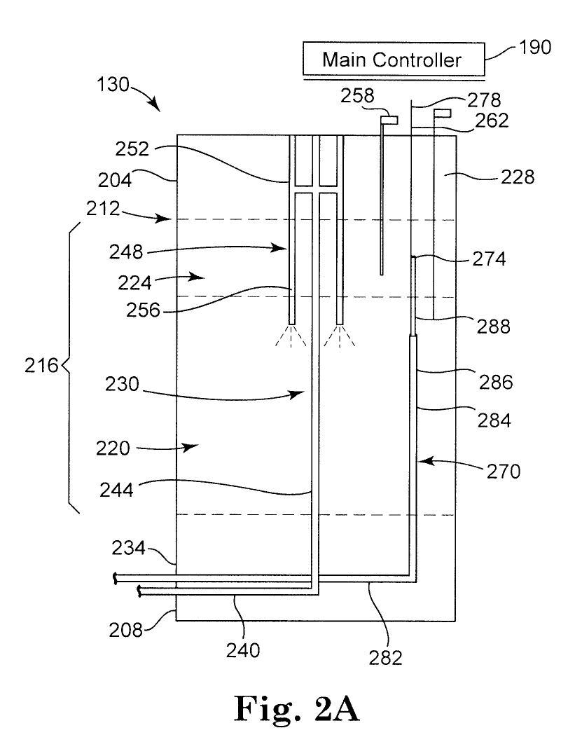

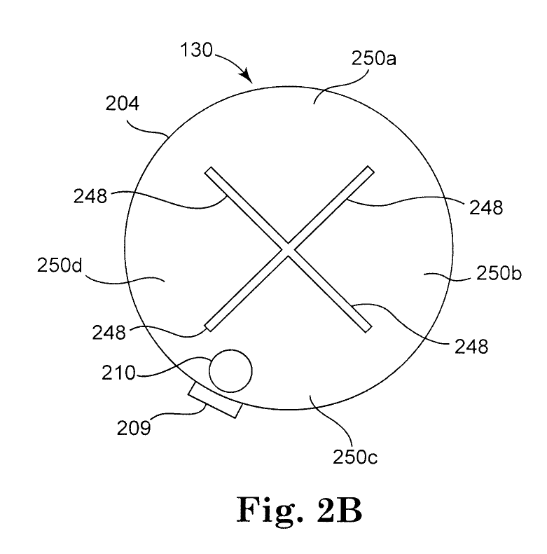

FIG. 2A is a side schematic view of the digestion tank 130 according to various embodiments of the present invention. FIG. 2B is a top schematic view of the digestion tank 130 shown in FIG. 2A. As shown in FIG. 2A, the digestion tank 130 includes a top end 204 and a bottom end 208. In some embodiments, the tank 130 may be generally cylindrical and may vary in size and volume depending upon the application. According to various embodiments, the tank 130 can be fabricated from fiberglass-reinforced plastic or stainless steel. In some embodiments, the tank 130 can be insulated. According to various embodiments, as shown in FIG. 2B, the tank 130 can include at least one ladder 209 and at least one man-way 210 for providing access to the digestion tank 130.

According to various embodiments of the present invention, as shown in FIG. 2A, the tank 130 contains a predetermined level 212 of a manure mixture 216 to be digested and a sufficient quantity of anaerobic bacteria capable of digesting the manure mixture 216 to produce biogas. The manure mixture 216 stratifies within the tank 130 to include a sludge layer 220 including solid elements and a liquid effluent layer 224 including liquid elements. The dashed lines shown in FIG. 2A generally indicate the level of each layer 220 and 224 within the manure mixture 216. It is generally understood that the level of each layer 220 or 224 may be higher or lower in the tank 130. Additionally, it is generally understood that the dashed lines represent fluid boundaries, rather than distinct boundaries between each layer 220 and 224. As shown in FIG. 2A, the sludge layer 220 is suspended in the middle of the tank, and may occupy up to about 50% of the total volume of the digestion tank 130. The liquid elements form a liquid effluent layer 224 on top of the sludge layer 220 and include digested material having a negligible amount of solids suspended in the liquid effluent layer 224. A headspace 228 is defined above the predetermined level 212 of the manure mixture 216 within the tank 130. Biogas produced during the digestion process is collected in the headspace 228.

According to various embodiments of the present invention, as shown in FIG. 2A, the digestion tank 130 includes a first inlet piping 230 coupled to a side 234 of the digestion tank 130 near its bottom end 208. Fresh influent enters the digestion tank 130 from the waste reception area 120, shown in FIG. 1, via the first inlet piping 230. According to one embodiment, the first inlet piping 230 includes a horizontal portion 240 and a main vertical portion 244. The main vertical portion 244 extends upwards in a vertical direction within the tank 130 and branches a plurality of arms 248. According to one exemplary embodiment, as best shown in FIG. 2B, the main vertical portion 244 may branch into four arms 248. According to other embodiments, the main vertical portion 244 may branch into any number of arms 248 to as to facilitate an efficient distribution of fresh waste into the tank 130. Each arm 248 includes an elbow portion 252 located above the predetermined level 212 of the manure mixture 216 within the tank 130 and a generally straight portion 256 that follows a downward path parallel to the main vertical portion 244. The elbow portion 252 is located above the predetermined level 212 of manure mixture 216 in the tank 130 creating a backflow barrier such that the tank 130 cannot drain itself through the first inlet piping 230. As best shown in FIG. 2B, the arms 248 divide the tank 130 into equal regions 250a-d such that the incoming influent is evenly distributed within the tank 130. Fresh influent enters the tank 130 via the first inlet piping 230 such that it is delivered below the predetermined level 212 of the manure mixture 216 already in the tank 130.

In some embodiments, the level of the manure mixture 216 inside the digestion tank 130 can be determined using two instruments. An upper level of the liquid effluent layer 224 is determined by an ultrasonic level detector 258 located inside of the digestion tank 130. An exemplary ultrasonic level detector is the Drexelbrook Ultrasonic Level Detector US11. It is generally recognized by those of ordinary skill in the art that other detectors capable of detecting the upper level of the liquid effluent layer 224 may also be employed. The level detector 258 detects and sends an input value indicative of the upper level of the liquid effluent layer 224 within the tank 130 to the main controller 190 where the input is stored and processed. In some embodiments, the main controller 190 sends an output command as appropriate determined by the input value received from the level detector 258.

The upper level of the sludge layer 220 within the tank 130 is determined by a sludge meter 262. An exemplary sludge meter is the Drexelbrook Clarifier Control System CCS 1160. Other meters capable of determining the upper level of the sludge layer 220 inside the digestion tank 130 may be employed. Like the level detector, the sludge meter 262 detects and sends an input value indicative of the upper level of the sludge layer 220 within the tank 130 to the main controller 190 where the value is stored and processed. In some embodiments, the main controller 190 may send an output command as appropriate determined by the input value received from the sludge meter 262.

According to various embodiments of the present invention as shown in FIG. 2A, the digestion tank 130 also includes a height adjustable valve 270 including an effluent intake end 274 and an actuator arm 278. The height adjustable valve 270 is coupled to and positioned within the tank 130 such that its intake end 274 is positioned and maintained within the liquid effluent layer 224. The sludge meter 262 detects the solids level within the tank 130 and communicates this to the main controller 190. In response to the level determinations made by the sludge meter 262, the main controller 190 sends an output command to the actuator arm 278 to adjust the position of the height adjustable valve 270. More particularly, the actuator arm 278 height is adapted to automatically adjust the position of the intake end 274 of the height adjustable valve 270 within the liquid effluent layer 224 in response to the level detections made by the sludge meter 262 to cause liquid effluent to be withdrawn from the tank 130 for recirculation.

According to some embodiments, the height adjustable valve 270 is coupled to the side 234 of the digestion tank 130 near its bottom end 208, and includes a horizontal portion 282 extending from the side 234 of the tank 130 and a vertical portion 284 rising vertically within the tank 130. The vertical portion 284 contains a larger diameter main portion 286 and a smaller diameter telescoping portion 288. The smaller diameter telescoping portion 288 is coupled to the actuator arm 278 located at the top end 204 of the tank 130 such that the actuator arm 278 is adapted to move the telescoping portion 288 in a vertical direction relative to the vertical portion 284 to adjust the overall height of the height adjustable valve 270. According to various embodiments of the present invention, the height adjustable valve 270 is coupled to the sludge meter 262 through the main controller 190 and the actuator arm 278 such that the position of its intake end 274 is automatically adjusted in response to the level of the sludge layer 220 detected by the sludge meter 262 such that the position of its intake end 274 is maintained within the liquid effluent layer 224 and above the level of the sludge layer 220. The height adjustable valve 270 can be positioned within the liquid effluent layer 224 such that the liquid effluent having a desired consistency is withdrawn from the tank and re-circulated, improving the overall efficiency of the digestion tank 130.

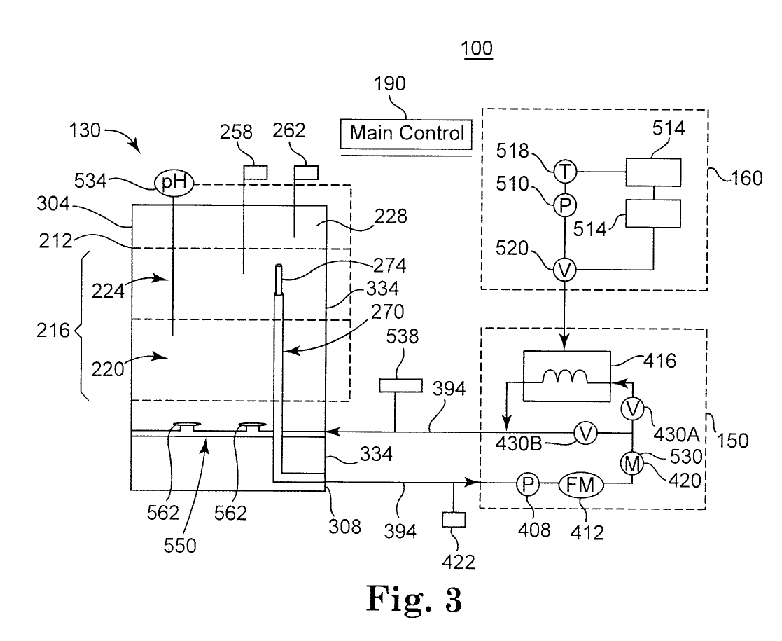

FIG. 3 is a detailed, schematic block diagram of a portion of the digestion system 100 including a digestion tank 130, a heat exchange system 150, and a water circulation system 160 according to various embodiments of the present invention. Together through the main controller 190, the heat exchange system 150 and the water circulation system 160 control and maintain the temperature of the manure mixture 216 within the digestion tank 130. According to various embodiments, as shown in FIG. 3, the heat exchange system 150 is fluidly coupled to the digestion tank 130 via at least one recirculation line 394. The recirculation line 394 passes through the heat exchange system 150 and returns to the digestion tank 130.

The heat exchange system 150 includes a recirculation pump 408, a flow meter 412, at least one heat exchanger 416, and a temperature monitor 420. When the level of the manure mixture 216 within the tank reaches a predetermined level as measured by the ultrasonic level detector 258 located within the digestion tank 130, recirculation of the liquid effluent commences upon receiving an output command from the main controller 190. In response to an output command received from the main controller 190, the recirculation pump 408 begins to draw the liquid effluent out of the digestion tank 130 via the height adjustable valve 270 and through the recirculation line 394 to the heat exchange system 150. The flow meter 412 detects the rate of liquid effluent flow from the digestion tank 130 to the heat exchange system 150, and sends an input value indicative of the flow rate to the main controller 190. The main controller 190 sends an output command, as appropriate, to the recirculation pump 408 to increase or decrease the liquid effluent flow rate in response to the input value received from the flow meter 412. According to further embodiments, the heat exchange system 150 includes an air compressor 422 for blowing out the recirculation line(s) when no flow is detected by the flow meter 412.

According to various embodiments of the present invention, the liquid effluent travels through recirculation line 394 and passes through the at least one heat exchanger 416 included within the heat exchange system 150. According to various embodiments of the present invention, the heat exchange system 150 can include more than one heat exchanger 416. The heat exchanger 416 can be any suitable heat exchanger as is known to those of ordinary skill in the art. According to various embodiments, the heat exchanger 416 is a dual pipe heat exchanger.

The temperature of the liquid effluent flowing through the recirculation line 394 from the digestion tank 130 through the heat exchanger 416 is measured by the temperature monitor 420. The temperature of the liquid effluent should be such that the temperature of the digestion tank 130 is maintained within a specified temperature range suitable for digestion. According to one embodiment the temperature of the liquid effluent should be within temperature range sufficient to maintain a temperature of the manure mixture 216 within the digestion tank 130 in a mesophilic temperature range. According to other embodiments, the temperature of the liquid effluent should be maintained within a temperature range sufficient to maintain a temperature of the manure mixture 216 within the digestion tank 130 in a thermophilic temperature range. In another embodiment, the flow rate of the liquid effluent passing through the heat exchanger 416 and the recirculation line 394 can be increased or decreased to maintain the temperature of the liquid effluent within a specified temperature range sufficient to maintain a temperature of the manure mixture 216 within the digestion tank in either a mesophilic temperature range or a thermophilic temperature range.

According to some embodiments, the recirculation line 394 can include one or more valves 430A and 430B adapted for allowing the liquid effluent to flow through or to bypass the heat exchanger 416. According to some embodiments, the valves 430A and B are motorized ball valves. The valves 430A and 430B are operated by the main controller 190. The main controller 190 receives an input value from the temperature monitor 420 indicative of the temperature of the liquid effluent flowing through the re-circulation line 394. In response to the input value received from the temperature monitor 420, the main controller 190 may send an output command to open or close the valves 430A and 430B as appropriate. When the temperature of the liquid effluent is below a target temperature range, the valves 430A and 430B can be operated through the main controller 190 such that the valve allows the liquid effluent to travel through the heat exchanger 416. When the temperature of the liquid effluent is within a target temperature range, the valves 430A and 430B can be closed via the main controller 190 so as to bypass the heat exchanger 416. For example, when valve 430A is open and valve 430B is closed, the liquid effluent flows through the heat exchanger 416 prior to being returned to the digestion tank 130 via the recirculation line 394. Conversely, when valve 430A is closed and valve 430B is opened, the liquid effluent flowing through the recirculation line 394 bypasses the heat exchanger 416 and returns directly to the digestion tank 130. According to a further embodiment, the flow rate of the liquid effluent flowing through the heat exchanger(s) 416 can be increased or decreased in response to the temperature detected by the temperature monitor 420.

Hot water is supplied to the heat exchanger system 150 from the water circulation system 160. The water circulation system 160 includes a water recirculation pump 510, one or more hot water heaters 514, a water temperature monitor 518, and a valve 520. The water recirculation pump 510 pumps hot water from the water heaters 514 to the heat exchanger(s) 416 where heat is transferred to the liquid effluent flowing through the recirculation line 394. According to some embodiments, the water circulation pump 510 is controlled through the main controller 190. The main controller 190 causes the water circulation pump 510 to increase or decrease the flow of hot water from the hot water heater(s) 514 to the heat exchanger(s) 416 in response to the temperature of the liquid effluent flowing through the recirculation line 394 detected by the temperature sensor 420. For example, if the temperature of the liquid effluent needs to be raised, the main controller 190 sends an output command to the water recirculation pump 510 to increase the flow rate of hot water to the heat exchanger(s) 416. Likewise, if the temperature of the liquid effluent is steady and/or within the desired temperature range, main controller 190 may send an output command to the water recirculation pump to decrease or stop the flow of hot water to the heat exchanger(s) 416. According to some embodiments, the water recirculation system 160 can include one or more valves 520 for bypassing the flow of hot water to the heat exchanger(s 416) in response to the temperature of the liquid effluent detected by the temperature sensor 420. The valves 520 can be actuated by the main controller 190 in response to an input value indicative of a temperature received by the main controller 190 from the temperature sensor 420. The water temperature of the water circulating through the hot water system 160 is monitored by the water temperature monitor 518 such that it remains within a temperature range sufficient to supply heat to the heat exchanger(s) 416.

According to various embodiments of the present invention, as shown in FIG. 3 the digestion system 100 also includes a pH monitoring system including a first pH probe 530 including the temperature monitor 420 discussed above, a second pH probe 534 including a temperature monitor, and a chemical feed line 538. The first pH probe 530 is located within the recirculation line 394 and determines the pH of the liquid effluent flowing through the recirculation line 394. The second pH probe 530 is located within the digestion tank 130 and determines the pH of the manure mixture 216 within the digestion tank 130. According to some embodiments, the first pH probe 530 is electrically coupled to the second pH probe 534 located within the digestion tank 130 via the main controller 190. The first and second pH probe 530 and 534 each provide an input value indicative of the pH level inside the digestion tank 130 or recirculation line 394. In response to the pH level determinations made by the first and second pH probe 530 and 534, the main controller 190 may send an output command to cause caustic lime to added to the liquid effluent via the chemical feed line 538 to adjust the pH of the liquid effluent prior to its re-introduction into the digestion tank 130 such that the pH of the manure mixture 216 within the digestion tank 130 is maintained within a specified pH range. According to one embodiment of the present invention, the pH of the manure mixture 216 within the digestion tank 130 is maintained within a mesophilic pH range. According to another embodiment, the pH of the manure mixture 216 within the digestion tank is maintained within a thermophilic range.

According to another embodiment of the present invention, the pH of the manure mixture 216 may be adjusted by controlling the flow rate of the liquid effluent flowing through the recirculation line 394 in conjunction with caustic lime added to the liquid effluent via the chemical feed line 538 to adjust the pH of the liquid effluent prior to its re-introduction into the digestion tank 130 such that the pH of the manure mixture 216 within the digestion tank 130 is maintained within a specified pH range. Controlling the pH of the manure mixture 216 by controlling the liquid effluent flow rate 216 through the recirculation line 394 may reduce and/or eliminate the need for caustic lime to be added to the liquid effluent prior to its return into the digestion tank 130.

As shown in FIG. 3, the heated, re-circulated effluent re-enters the digestion tank 130 via a second inlet piping 550 coupled to a side 334 of the of the tank 130 located near its bottom end 308. The second inlet piping 550 extends parallel to a bottom of the tank 130 and includes a plurality of orifices 562. Heated, re-circulated liquid effluent is forced through orifices 562 and re-collects within the tank 130. On one embodiment, the re-circulated effluent is sprayed upwards towards suspended sludge layer 220. The upward spray pattern provided by the second inlet piping 550 facilitates interaction between the liquid and the solids elements of the manure mixture 216 by facilitating mixing of the manure mixture 216.

According to one embodiment, the second inlet piping 550 includes a Tonka Inlet System manufactured by the Tonka Equipment Company of Plymouth, Minn. A Tonka inlet system is circular and includes interconnecting circles. The re-circulated effluent enters and is forced around an outer circle with openings into an inner cavity. The circular pathway is configured such that any solids are macerated prior to re-entering the digestion tank. The opening in the center cavity allows the liquid effluent to re-enter the digestion tank. The circular configuration creates a tornado-like spray system. The Tonka inlet system facilitates formation of the suspended sludge layer 220 in the middle of the tank. The upward spray pattern maintains the suspended sludge layer and facilitates contact interaction between the liquid and solids elements of the manure mixture 216 by mixing the manure mixture 216.

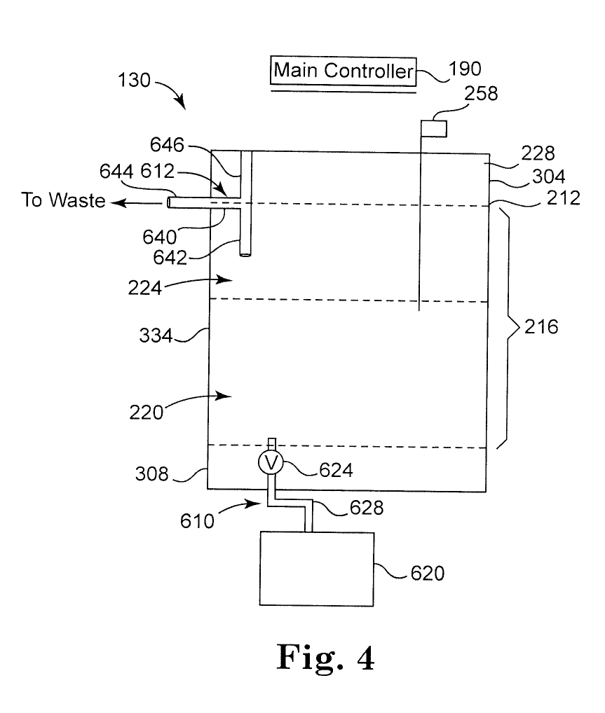

FIG. 4 is a side schematic view of a digestion tank 130 according to various embodiments of the present invention. As shown in FIG. 4, the digestion tank 130 includes a sludge release mechanism 610 and a liquid effluent release mechanism 612. Like the other components of the system the sludge release mechanism is controlled through the main controller 190, introduced in FIG. 1. According to various embodiments, digested effluent can be released from the tank 130 via the sludge release mechanism 610 and/or the liquid effluent release mechanism 612.

The sludge release mechanism 610 is located at the bottom 308 of the tank 130 and releases effluent in the form of sludge to a waste reclamation area 620. The sludge release mechanism 610 includes a motorized valve 624 adapted to release sludge through piping 628 to the waste reclamation area 620. According to some embodiments, the motorized valve 624 is a motorized ball valve. It is generally recognized by those of skill in the art that other motorized valves may be used. The motorized valve 624 is controlled by the main controller 190 and is adapted to actuate between an open and a closed position in response to the level of the sludge layer 220 detected by the sludge meter 262 located within the digestion tank 130. For example, according to one embodiment, when the volume of the sludge layer 220 as determined by the sludge meter 262 increases to a volume greater than fifty percent of the total volume of the digestion tank 130, the motorized valve 624 is actuated by the main controller 190 to open and release sludge from the bottom of the tank 308 to the waste reclamation area 620. The valve 624 is actuated by the main controller 190 to close when the volume of the sludge layer 220 reaches a predetermined level equal to or less than fifty percent of the total volume of the digestion tank 130. According to other embodiments of the present invention, if the solids level does not rise above fifty percent of the total volume of the tank 130, the motorized valve 624 can be operated on a timing mechanism to periodically release sludge from the bottom of the tank 130 over a specified time period.

According to another embodiment, fresh influent can be transferred from the waste reception area 120 to the digestion tank 130 in a batch-wise process or a continuous process. According to one embodiment, liquid effluent is released from the digestion tank 130 via the liquid effluent release mechanism 612 in equal proportion to the amount of fresh influent transferred to the digestion tank 130 from the waste reception area 120.

The liquid effluent release mechanism 612 is located near the top 204 of the digestion tank 130 and releases effluent from the tank 130 in the form of a liquid. According to one embodiment, as shown in FIG. 4, the liquid effluent release mechanism 612 includes piping 640 having a “T” configuration. The piping includes first, second and third portions 642, 644, and 646. The first portion 642 of the piping 640 follows a path parallel to a side wall 334 of the tank 130 and extends into the liquid effluent layer 224 of the manure mixture contained within the tank 130. The second portion 644 of the piping 640 extends parallel to the bottom 308 of the tank and is coupled to the side wall 334 of the tank 130. The second portion 644 of the piping 640 is positioned at approximately the same elevation as the level of the liquid effluent layer 224 inside of the tank 130. When fresh influent is added to the tank 130, the level of the liquid effluent layer 224 in the digestion tank 130 rises in proportion to the amount of influent added. The first portion 642 of the piping 640 extending into the liquid effluent layer 220 experiences the same increases in volume. When the level of the mixture in the tank rises above the level of the second portion 644 of the piping 640, it is released out of the digestion tank 130 through the second portion 644 of the piping 640. According to one embodiment the second portion 644 is coupled to effluent waste piping provided external to the tank 130. The piping 640 transports the released effluent to a waste storage area or solids processing area. The third portion 646 of the piping 640 extends to the top 304 of the tank 130 and is provided to remove debris blocking the piping 640 or any external piping coupled to the second effluent release mechanism 612. Additionally, the third portion 646 facilitates the removal of liquid samples. The location of the liquid effluent release mechanism 612 in combination with its T-shaped configuration having a first portion extending into the liquid effluent layer and a third portion extending above a plane of the liquid effluent layer provides a barrier preventing biogas collected in the headspace 228 of the tank 130 from escaping through the liquid effluent release mechanism.

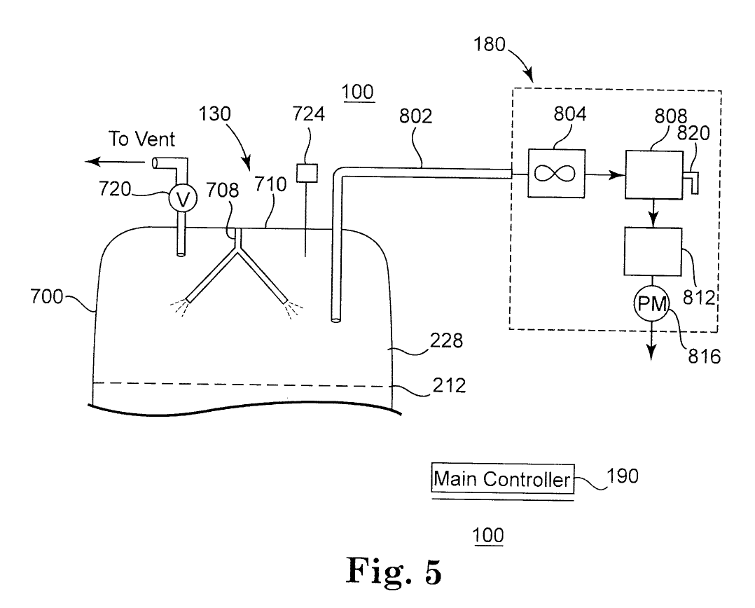

FIG. 5 is a schematic view of a portion of the digestion system 100 including a digestion tank 130 including a top portion 700 and a biogas conditioning system 180. Biogas accumulates in the headspace 228 defined in the top portion 710 of the digestion tank 130. A spray system 708 is attached to the top 710 of the tank 130 above the predetermined level of liquid 212 in the tank 130 to spray down any foam built up during the mixing process. The digestion tank 130 also includes a vacuum/pressure release valve 720 including a pressure sensor 724 coupled to the top 710 of the digestion tank 130. The pressure release valve 720 is a mechanical valve configured to open when the pressure of the biogas collected in the headspace 228 of the digestion tank 130 reaches a predetermined value to vent a portion of the biogas from the digestion tank 130 as needed for safety purposes.

Biogas collected in the headspace 228 of the digestion tank 130 contains impurities along with methane gas. These impurities are mostly hydrogen sulfide and water vapor. The biogas conditioning system 180 is used to remove the impurities including the water vapor from the biogas. The biogas conditioning system 180 is fluidly coupled to the digestion tank 130 via an insulated gas line 802, and includes a positive displacement blower 804, a hydrogen sulfide sponge 808 including gas conditioning media, moisture knockout 812, and a flow meter 816. It is generally recognized by those of skill in the art that any commercially available biogas conditioning equipment may be used to condition the biogas.

The removal of the biogas from the headspace 228 of the digestion tank 130 is facilitated by the positive displacement blower 804 and is controlled by the main controller 190. The size of the displacement blower 804 is variable to the amount of waste being treated. The blower 804 is operated by the main controller 190 such that the removal rate of the biogas can be increased or decreased in response to the pressure inside the digestion tank 130 detected by the pressure sensor 724. The gas travels through an insulated gas line 802 that extends from the top 710 of the tank 130 to the hydrogen sulfide sponge 808 and moisture knockout 812. The size of the hydrogen sulfide sponge 808 and moisture knockout 812 is selected to accommodate the amount of biogas produced by the digestion system 100.

The biogas enters the hydrogen sulfide sponge 808 where the hydrogen sulfide in the gas is removed. The hydrogen sulfide sponge 808 includes biogas conditioning media for removing impurities present in the biogas. The size of the sponge 808 and amount of biogas conditioning media is determined by the hydrogen sulfide content of the biogas. According to one embodiment, the biogas conditioning media includes woodchips impregnated with iron shavings. The hydrogen sulfide particles present in the biogas are attracted to the iron shavings and bind to the woodchips where a chemical reaction occurs converting the hydrogen sulfide to water. The water exits the sponge 808 via a drip leg 820. The biogas then travels through the insulated gas line 802 to the moisture knockout 812 where water vapor present in the biogas is precipitated from the biogas.

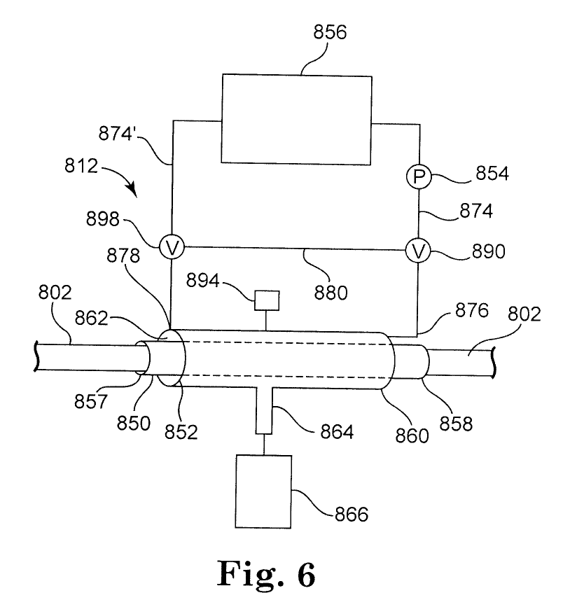

FIG. 6 is a schematic view of the moisture knockout 812 according to one embodiment of the present invention. Biogas removed directly from the digestion tank 130, as described above, has approximately the same temperature as the manure mixture inside of the digestion tank 130. According to one embodiment of the present invention, the temperature of the biogas ranges from about 60 to about 120 degrees Fahrenheit. According to another embodiment, the temperature of the biogas ranges from about 120 to about 160 degrees Fahrenheit. The moisture knockout 812 lowers the temperature of the biogas and causes water vapor to precipitate from the biogas. The size of the moisture knockout 812 is variable with the amount of waste being treated.

According to one embodiment, as shown in FIG. 6, the moisture knockout 812 includes a first pipe 850 of a smaller diameter contained within a second pipe 852 of a larger diameter such that a space is defined between the first and second pipes 850 and 852. The pipes 850 and 852 can be constructed of a variety of materials suitable for transporting biogas including fiberglass-reinforced plastic or stainless steel. According to one embodiment, a diameter of the first pipe 850 is substantially equal to the diameter of the insulated gas line 802 connecting the digestion tank 130 to the biogas conditioning system 180 including the moisture knockout 812. A cold water circulation pump 854 pumps water from a cold water reservoir 856 through the space defined between the larger diameter pipe 852 and the smaller diameter pipe 850 to precipitate water from the biogas.

The first, smaller diameter pipe 850 is contained within the second, larger diameter pipe 852 and includes a first end 857 and a second end 858. According to some embodiments, as shown in FIG. 6, the first and second ends 857 and 858 of the smaller diameter pipe 850 extend beyond first and second ends 860 and 862 of the larger diameter pipe 852, and are coupled to the insulated gas line 802 located on either side of the moisture knockout 812. Additionally, according to some embodiments, the smaller diameter pipe 850 includes a drip leg 864 having a “T” shaped configuration extending to a collection reservoir 866 located external to the moisture knockout 812. Water precipitated from the biogas traveling through the smaller diameter pipe 850 is released from the moisture knockout 812 through the drip leg 864 and into the collection reservoir 866.

The larger diameter second pipe 852 creates an air tight seal around the smaller diameter pipe 850. The larger diameter pipe 852 is connected via piping 874 to the cold water reservoir 856. Additionally, the larger diameter pipe 852 includes first and second couplings 876 and 878 located on the first and second ends 860 and 862 of the pipe 852. The couplings 876 and 878 are connected to additional piping 874 forming a loop 880 with the cold water reservoir 856. Cold water is pumped from the reservoir 856 into the first end 860 of the larger diameter pipe 852. The water fills the larger diameter pipe 852 and surrounds the smaller diameter pipe 850 before exiting through the second coupling 878 located on the second end 862 of the larger diameter pipe 852. Once the water exits the moisture knockout 812 it is returned to the reservoir via the loop 880.

According to some embodiments of the present invention, the moisture knockout 812 includes a temperature probe 894 located within the larger diameter pipe 852. According to one embodiment, the temperature probe 894 detects and monitors the temperature of the water flowing through the moisture knockout 812. As shown in FIG. 6, the moisture knockout 812 can also include a first valve 896 and a second valve 898 coupled to the cold water piping 874. The valves 896 and 898 can be used to direct the flow of the cold water through or to bypass the moisture knockout 812. In one embodiment, the valves 896 and 898 can be actuated by the main controller 190 in response to the temperature detected by the temperature probe 894. In other embodiments, the valves 896 and 898 can be actuated by the main controller in response to the temperature of the biogas as detected by the flow meter 816 (FIG. 5), as will be described in further detail below. According to one further embodiment, the circulation pump 854 may be switched off by the main controller 190 in response to the temperature determinations made by the temperature probe 894 or in response to the temperature determinations made by the flow meter 816.

Once the biogas has passed through the biogas conditioning system 180, it flows through the flow meter 816, as shown in FIG. 5, prior to its use in biogas applications. The meter 816 reads the flow rate and temperature of the passing biogas. According to some embodiments, the meter 816 is a Fox FT2 flow meter. The information from the meter 816 is sent to the main controller 190 and used to calculate a number of values and to adjust the overall processing parameters as necessary. For example, according to one embodiment, biogas temperature determinations made by the flow meter 816 can be used to control the flow of cold water passing through the moisture knockout 812, affecting the precipitation of impurities from the biogas via the main controller 190. For example, in some embodiments, the valves 896 and 898 can be actuated by the main controller 190 in response to the temperature of the biogas detected by the flow meter 816. When the temperature of the biogas flowing through the moisture knockout 812 is less than a predetermined value, the first valve 896 connected to the piping 874 leading from the cold water reservoir 856 to the larger diameter pipe 852 is closed and the second valve 898 is opened such that the water bypasses the moisture knockout 812 returns to the cold water reservoir 856 via the feedback loop. In another example, when the temperature of the biogas is greater than a predetermined valve, the first valve 896 is opened allowing water to flow from the cold water reservoir 856 to the moisture knockout 812, and the second valve 898 is closed. This process continues until the temperature as determined by the flow meter 816 indicates that the temperature of the biogas flowing through the biogas conditioning system 180 is within a predetermined temperature range suitable for precipitation of water from the biogas. In other embodiments, the temperature and/or flow rate determinations made by the meter 816 can be used to control the flow rate of the biogas flowing through the biogas conditioning system 180. In a further embodiment, temperature and/or flow rate determinations made by the meter 816 communicated to the main controller 190 can be used to control other process variables to maximize the efficiency production of biogas by the system.

After passing through the meter 816, a majority of the biogas can be transferred to a utilization point for conversion into electricity, offsetting natural gas or propane consumption, or cogeneration. According to some embodiments, the conditioned biogas can be used to provide energy to the digestion system 100 making the digestion system 100 self-sustaining.

We claim:

1. A digestion system for converting a manure mixture comprising a liquid effluent layer having a liquid level and a sludge layer having a solids level, the system comprising:
a digestion tank including:
a first end and a second end, a headspace defined above the liquid level within the tank, and a height adjustable valve including an intake end positioned within the liquid effluent layer and configured to withdraw liquid effluent from the liquid effluent layer and out of the digestion tank;
a heat exchange system fluidly coupled to the digestion tank, wherein liquid effluent withdrawn from the liquid layer is passed through the heat exchange system for heating before being returned to the digestion tank;
a water recirculation system fluidly coupled to the heat exchange system for providing heat to the heat exchange system;
a biogas collection system for regulating a level of biogas collected in the headspace of the digestion tank;
a biogas conditioning system for processing the biogas, and
a main controller for controlling the height adjustable valve, the heat exchange system, the water recirculation system, the biogas collection system, and the biogas conditioning system.

2. The digestion system according to claim 1, wherein the height adjustable valve includes a telescoping portion adapted to move in a vertical direction between at least a first position and a second position such that the intake end is maintained within the liquid effluent layer and above the sludge layer.

3. The digestion system according to claim 1, further comprising a sludge meter located within the tank, the sludge meter adapted to determine the solids level within the digestion tank.

4. The digestion system according to claim 3, wherein the height adjustable valve is coupled to the sludge meter through the main controller such that the main controller automatically adjusts the position of the intake end of the height adjustable valve in response to the solids level determined by the sludge meter such that the intake end is maintained within the liquid effluent layer.

5. The digestion system according to claim 1, further comprising a pH control system coupled to and controlled through the main controller, the pH control system including a first pH probe located within the digestion tank and adapted to determine a pH of the mixture within the digestion tank, a second pH probe located within a recirculation line fluidly connecting the heat exchange system to the digestion tank, the second pH probe adapted to determine a pH of the liquid effluent returning to the digestion tank from the heat exchange system via the recirculation line, and a chemical feed coupled to the recirculation line adapted to deposit an amount of a pH altering compound into the liquid effluent flowing through the recirculation line in response to the pH of the mixture within the tank determined by the first pH probe and the pH of the liquid effluent flowing through the recirculation line determined by the second probe such that the pH of the mixture within the digestion tank is maintained within a specified pH range for digestion.

6. The digestion system according to claim 1, further comprising a pH control system coupled to and controlled through the main controller, the pH control system including: a first pH probe located within the digestion tank and adapted to determine a pH of the mixture within the digestion tank and a second pH probe located within a recirculation line fluidly connecting the heat exchange system to the digestion tank, the second pH probe adapted to determine a pH of the liquid effluent returning to the digestion tank from the heat exchange system via the recirculation line, and wherein the main controller is adapted to adjust a flow rate of the liquid effluent flowing through the recirculation line in response to the pH of the mixture within the tank determined by the first pH probe and the pH of the liquid effluent flowing through the recirculation line determined by the second probe such that the pH of the mixture within the digestion tank is maintained within a specified pH range for digestion.

7. The digestion system according to claim 1, further comprising a second inlet piping adapted to spray liquid effluent returning to the tank from the heat exchange system in an upwards direction so as to effect mixing of the manure mixture within the tank.

8. The digestion system according to claim 1, wherein an amount of fresh waste transferred to the digestion tank is substantially equal to an amount of digested waste released from the digestion tank.

9. The digestion system according to claim 1, wherein an amount of fresh waste is transferred to the digestion tank in a continuous process controlled by the main controller.

10. The digestion system according to claim 1, wherein an amount of fresh waste is transferred to the digestion tank in a batch-wise process controlled by the main controller.

11. The digestion system according to claim 1, wherein liquid effluent returning from the heat exchange system to the digestion tank mixes the manure mixture with the tank while maintaining a temperature and a pH of the manure mixture within the tank within a mesophilic range.

12. The digestion system according to claim 1, wherein liquid effluent returning from the heat exchange system to the digestion tank mixes the manure mixture within the tank while maintaining a temperature and a pH of the manure mixture within the tank within a thermophilic range.

13. The digestion system according to claim 3, further comprising a sludge release mechanism coupled to the second end of the tank and in communication with the sludge meter through the main controller, the sludge release mechanism including a valve adapted to be actuated by the main controller between an open position and a closed position in response to the solids level detected by the sludge meter, wherein the sludge release mechanism releases effluent in the form of sludge from the second end of the tank.

14. The digestion tank assembly according to claim 1, wherein the height adjustable valve includes a horizontal portion extending from the side of the tank and a vertical portion rising vertically within the tank, the vertical portion comprising a larger diameter main portion and a smaller diameter telescoping portion, wherein the smaller diameter telescoping portion moves in a vertical direction relative to the larger main portion to adjust a position of the height adjustable valve within the tank.

15. The system according to claim 1, wherein the digestion tank further comprises a first inlet pipe coupled to a side wall of the digestion tank, the inlet pipe comprising a main vertical portion extending in a vertical direction towards the second end of the tank and a plurality of arms branching from the main vertical portion each arm comprising an elbow portion located above the predetermined level of the manure mixture within the tank and an arm portion parallel to the main vertical portion of the inlet pipe wherein the influent is deposited below the predetermined level of manure mixture within the tank, the arms dividing the tank into equal regions such that influent is evenly distributed within the tank.

16. The system according to claim 15, wherein the elbow portion breaks a plane of a level of liquid effluent within the tank so as to prevent backflow.

17. A digestion tank assembly for converting a manure mixture including a liquid effluent layer having a liquid level and a sludge layer having a solids level to biogas comprising:
a digestion tank including a first end, a second end and a headspace defined above the liquid level wherein biogas collects in the headspace; and
a height adjustable valve including an intake end configured to withdraw liquid effluent out of the digestion tank and adapted to move in a vertical direction between at least a first position and a second position such that the intake end is maintained within the liquid effluent layer and above the sludge layer within the digestion tank.

18. The digestion tank assembly according to claim 17 further comprising a sludge meter adapted for detecting the solids level within the tank.

19. The digestion tank assembly according to claim 18, wherein a position of intake end is adjusted by the main controller in response to the solids level within the tank determined by the sludge meter.

20. The digestion tank according to claim 18, further comprising a sludge release mechanism coupled to the second end of the tank and in communication with the sludge meter through the main controller, the sludge release mechanism including a valve adapted to be actuated by the main controller between an open position and a closed position in response to the solids level detected by the sludge meter, wherein the sludge release mechanism releases effluent in the form of sludge from the second end of the tank.

21. The digestion tank assembly according to claim 16, wherein the height adjustable valve includes a horizontal portion and a vertical portion rising vertically within the tank, the vertical portion comprising a larger diameter main portion and a smaller diameter telescoping portion, wherein the smaller diameter telescoping portion moves in a vertical direction relative to the larger main portion to adjust a position of the height adjustable valve within the tank.

22. The digestion tank assembly according to claim 17 further comprising a ultrasonic level detector for monitoring an amount of the liquid effluent within the tank.

23. The digestion tank assembly according to claim 17, further comprising a first inlet pipe coupled to a side wall of the digestion tank, the inlet pipe comprising a main vertical portion extending in a vertical direction towards the second end of the tank and a plurality of arms branching from the main vertical portion each arm comprising an elbow portion located above the predetermined level of the manure mixture within the tank and an arm portion parallel to the main vertical portion of the inlet pipe wherein the influent is deposited below the predetermined level of manure mixture within the tank, the arms dividing the tank into substantially equal regions such that influent is evenly distributed within the tank.

24. The digestion system according to claim 17, further comprising a second inlet piping adapted to spray liquid effluent entering the tank in an upwards direction so as to effect mixing of the manure mixture within the tank

25. The digestion system according to claim 17, further comprising a liquid effluent release coupled to a sidewall near a top end of the tank, the liquid effluent release mechanism configured to release digested liquid effluent in an amount equal to an amount of fresh effluent transferred to the digestion tank.

26. The digestion system according to claim 25, wherein the liquid effluent release mechanism comprises piping having a T-shaped configuration including a first portion extending into the liquid effluent layer, a second portion coupled to the sidewall of the tank and configured to release digested liquid effluent, and a third portion extending upwards and coupled to the top end of the tank, the third portion configured to provide access for maintenance and sample removal.

27. A process for converting agricultural waste to biogas comprising:
a) transferring fresh waste from a waste reception area to an anaerobic digestion tank including a sufficient quantity of anaerobic bacteria to digest the waste to produce a manure mixture having a predetermine level and including a liquid effluent layer having a liquid level and a sludge layer having a solids level and biogas;
b) determining the solids level of the sludge layer;
c) maintaining an intake end of a valve configured to withdraw liquid effluent from the digestion tank within the liquid effluent layer and above the sludge layer;
d) withdrawing liquid effluent from the liquid effluent layer;
e) recirculating the liquid effluent by passing the liquid effluent through a heat exchange system fluidly coupled to the digestion tank and returning the liquid effluent to the digestion tank; and
f) spraying the returning liquid effluent in an upwards direction to mix the manure mixture within the tank.

27. The method according to claim 27, further comprising releasing digested liquid effluent from the digestion tank in an amount equal to an amount of fresh waste transferred to the digestion tank.

28. The method according to claim 27, further comprising heating the liquid effluent passing through the heat exchange system to produce heated liquid effluent.

29. The method according to claim 27, further comprising maintaining a pH and a temperature of the manure mixture within a mesophilic pH range.

30. The method according to claim 27, further comprising maintaining a pH and a temperature of the manure mixture within a thermophilic range.

31. The method according to claim 27, further comprising controlling a flow rate of the liquid effluent returning to the digestion tank to maintain a pH of the manure mixture within a pH range suitable for digestion.

32. The method according to claim 27, further comprising controlling a flow rate of the liquid effluent passing through the heat exchange system to maintain a temperature of the manure mixture within the digestion tank within a temperature range suitable for digestion.

33. The method according to claim 27, further comprising controlling a flow rate of the liquid effluent returning to the digestion tank to control mixing of the manure mixture within the tank.
